(19) United States
(12) Patent Application Publication
(10) Pub. No.: US 2016/0228130 A1
(43) Pub. Date: Aug. 11, 2016

(54) Surgical Drill Bit

(71) Applicant: Cutting Edge Medical LLC, Louisville, KY (US)

(72) Inventors: Franc Koljaka, Louisville, KY (US); David Y Phelps, Louisville, KY (US)

(73) Assignee: CUTTING EDGE MEDICAL LLC, Louisville, KY (US)

(21) Appl. No.: 14/617,148

(22) Filed: Feb. 9, 2015

(51) Int. Cl. A61B 17/16

(52) U.S. Cl. CPC A61B 17/1615; A61B 17/1617

(57) ABSTRACT

A surgical drill bit is formed by a cylindrical body that comprises a distal fluted drill section, a fluted midsection and a proximal drive transmission shank. The distal end of the drill section forms the drill point of the surgical bit and comprises at least two opposed cutting edges that extend from the tip of the drill point to the leading edge of a land defined by the outer surface of a respective drill point flute. The outside diameter of the midsection is less than the outside diameter of the drill section. In cross section the flute consists of a land radially extending from an enlarged central web. A small conical tip is concentrically located on the drill point.

### FIELD OF THE INVENTION

This invention relates to material drilling tools and more particularly to surgical drill bits.

### BACKGROUND OF THE INVENTION

Drilling of bone is a very common operation in many fields of surgery such as orthopedics, plastic surgery, neurosurgery and the like. In the field of dentistry the fixing of implants requires drilling of bone. Generally the purpose of bone drilling is to create a bore or socket to accommodate a screw or other implant device. The bore or socket is formed using a drill bit secured in a drill mechanism which is usually powered by an electric motor.

The drilling of bone presents several problems for the surgeon. The chief problem being an increase in temperature caused by friction during the drilling process. Other risks include drill bit failure inside the bone that sometimes must be left in the bone because it cannot be extracted. Yet another problem is so called “bit walking” that is difficulty in holding the drill bit at the desired location, especially when drilling into bone at an acute angle. Bit walking presents a risk of severing soft tissue such as muscle, blood vessels and nerves. In addition it can also result in a bore larger than the diameter of the bit so that the implant is loosely fitting causing the fixture or implant to subsequently fail that requires the patient to undergo the expense and discomfort of another procedure.

Multiple studies have shown that the rise in temperature of bone during drilling can cause thermonecrosis of the bone resulting in the subsequent failure of the installed implant. The threshold temperature is 47° C. and temperatures above this value may cause osteonecrosis. The time at which bone is held at a temperature value above the threshold is another very important parameter that can affect the response of bone to the drilling operation. It widely accepted in the surgical field that the maximum holding time above 47° C. is 1 minute. Maintaining bone above these levels can result in osteonecrosis with irreversible bone damage and detrimental effects on the solidarity of the implant.

In the drilling process the surgical bit is subjected to torsional and bending loads that can produce significant shear and normal stresses. Surgical drill bits are available in a variety of diameters ranging from 0.76 mm to 17 mm but the vast majority falls in the range of 2 mm and 4 mm. The small diameter of most surgical drill bits coupled with the weakening of the cross section due to the flute shape and the stresses increase the chances of bit failure during the drilling operation. Many times due to the complexity of the operation and the limited time and space, the surgeon may decide not to remove the broken part of the bit but to leave it in the bone.

The round shape and the smooth surface of the bone, as well as the presence of liquids, can complicate the drilling start, especially if drilling at a small acute angle. In this case the bit may wander from its starting position which, as discussed above, may result in damaging soft tissue and producing a bore too large for the implant.

The design of the flute and drill point of the bit as well as the condition of the surface of the bit can enhance or reduce the negative factors discussed above. The increase in bone temperature is an effect of the heat generated during the drilling process that in turn is the result of the friction forces between the bit and the bone.

The present invention offers an improved surgical bone drill over conventional surgical bone drills.

### OBJECTS OF THE INVENTION

It is an object of the present invention to provide a surgical drill bit, particularly for drilling bone, which develops less heat buildup during the drilling operation than conventional drill bits.

Another object of the invention is to provide a drill bit that is less subject to failure due to torsional and flexural stress developed during a drilling operation.

Still another object of the invention is to provide a surgical drill bit point that is less subject to wander during the start of a drilling operation.

### SUMMARY OF THE INVENTION

The present invention relates to a new and improved surgical drill bit designed minimize the deficiencies found with prior art surgical drill bits, such as the buildup of heat in the object being drilled. In addition the surgical drill bit of the present invention has improved resistance to torsional and flexural stress and thus is less likely to fail during a drilling procedure. The drill point of the surgical drill bit of the invention is designed to maintain the drill point at the desired position during startup.

The drill bit is a cylindrical body that comprises a drill section, a midsection and a drive transmission shank. The drive transmission shank defines the proximal end of the bit and is adapted for attachment to a driving hand piece of conventional design. The surface of the drive transmission shank is smooth and normally is not in contact with bone.

The drill section defines the distal end portion of the surgical bit and comprises one or more flutes which define radially, helically extending lands along the length of the drill section and a drill point. The distal end of the drill section forms the drill point of the surgical bit and comprises at least two opposed cutting edges that extend from the tip of the drill point to the leading edge of a land defined by the outer surface of a respective drill point flute. The angle between the opposed cutting edges may be between 90° and 180°. The diameter of the drill section at the extending end of the lands forms the outside diameter of the bit and defines the diameter of the bore formed by the bit. The width of the land of the drill section at the outer edge is held at a minimum for minimum bearing surface area to reduce contact between the surface and the wall of the bore being formed. A portion of the cutting edges adjacent the tip may be concave to form a centric starter tip to aid in maintaining the position of the drill bit upon startup.

The midsection is defined by one or more helical flutes that extend radially from a central web and extend radially, spirally to the drill section. The midsection is adapted to remove bone fragments during the drilling operation and is not necessarily involved in the actual drilling. The outside diameter of the midsection is less than the outside diameter of the drill section so that the edges of the flute lands are not in contact with the wall of the bore during the drilling operation.

In cross section each flute in both the drill section and the midsection defines a radially extending land and an enlarged central web of at least 40% of the outside diameter of the bit at the drill section. The land radially decreases in width to the outer surface of the land.

The condition of the surface of the drill bit is a factor in heat buildup. The surface of the bit may be polished, electro polished, fine ground or coated with a low friction coating.

These and other advantages of the present will be apparent from the following description of the invention and the attendant drawings.

### BRIEF DESCRIPTION OF THE DRAWINGS

FIG. 1 is a perspective view of a prior art surgical drill;

FIG. 2 is a sectional view of the drill bit of FIG. 1 taken along line 2-2;

FIG. 3 is a perspective view of a two fluted surgical drill bit in accordance with the invention;

FIG. 4 is a sectional view of the drill bit of FIG. 3 taken along line 4-4;

FIG. 5 is a sectional view of the drill bit of FIG. 3 taken along line 5-5; and

FIG. 6 is an enlarged perspective view of the drill bit of FIG. 3 particularly illustrating a preferred drill point in accordance with the invention.

### DESCRIPTION OF THE PREFERRED EMBODIMENTS OF THE INVENTION

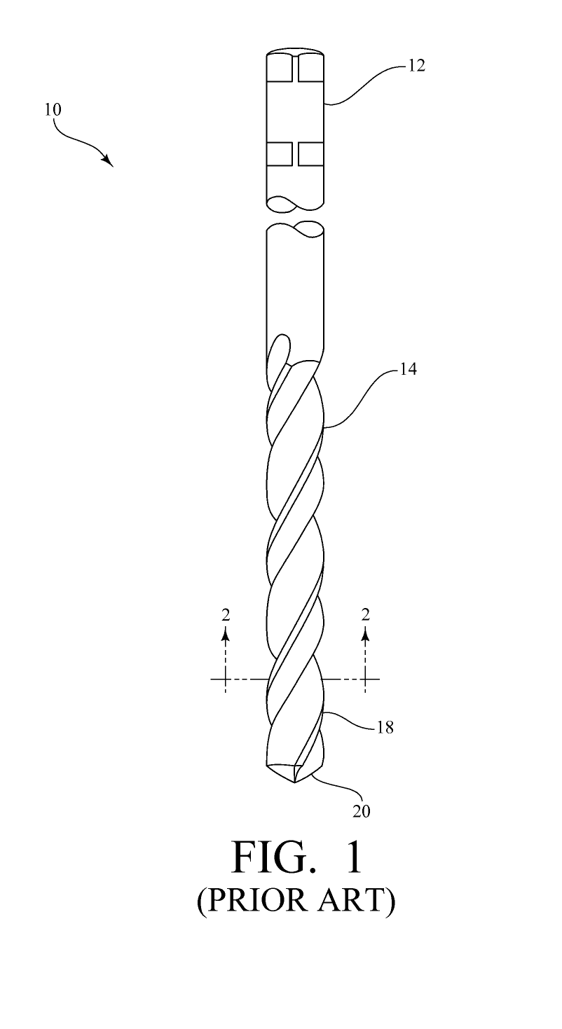

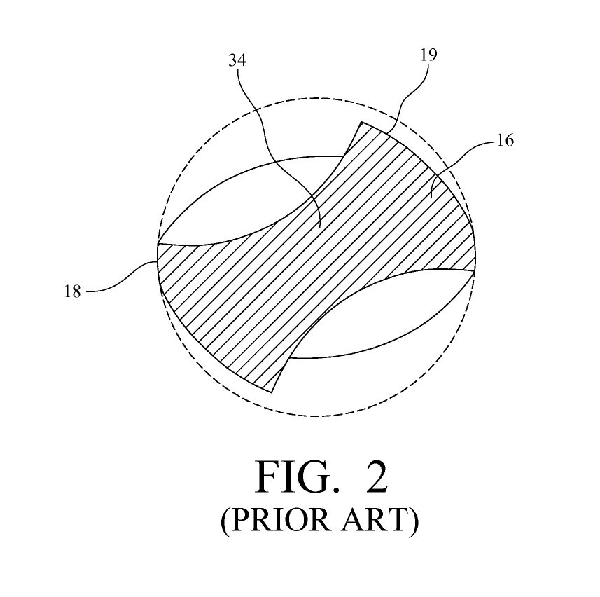

As illustrated in FIG. 1 and FIG. 2, a conventional prior art surgical drill bit shown generally as 10 consists of a drive section 12 for attachment to a power tool and a fluted section 14 comprising two opposed helical flutes comprising lands 16 extending radially to the axis of the drill bit. As shown most clearly in FIG. 2 the leading edges 18 of the lands 16 are cutting edges for contacting and cutting away bone being drilled. The trailing portion 19 of each edge is sloped slightly inwardly to define body clearance between the wall of the bore and the surface 19 for the collection and removal of bone chips. At the distal end of the drill bit the opposed cutting edges 18 are inwardly sloped to form a drill point 20. The outside diameter of the fluted section 14 is maintained at nominal value along the length of the fluted section and the edges 18 of the lands 16 are in contact with wall of the bore being drilled. The contact between the cutting edges 18 and bone being drilled as well as contact between the bone chips created by the drilling operation and the trailing portion 19 generates a substantial amount of heat in the bone that, as mentioned above, can rapidly reach the limiting temperature of about 47° C. If held at 47° C. for more than one minute substantial damage and even necrosis of the bone will result.

Accordingly, the surgeon must exercise extreme care when drilling bone to ensure that the temperature of the bone does not exceed about 47° C. for more than one minute. This may be accomplished by drilling for short periods of time with intervals between drilling periods to allow the bone to cool. Alternatively, a cooling fluid may be introduced to the drilling site to reduce the buildup of bone temperature. Some prior art drill bits are provided with a coolant channel (not shown) that opens to the drill 20 point and its use is commonly understood by those skilled in the art.

Surgical drill bits must also withstand substantial torsional and flexural stress applied during the drilling procedure. However surgical drill bits are normally small, on the order of 1 mm to 5 mm in cross section and due to the flutes the core of the drill body is even smaller in cross section. Thus breaking or bending of the drill bit may be a frequent occurrence. In some cases the broken portion of the drill bit cannot be recovered and must be left in the bone. As illustrated in FIG. 2 the area between the lands 16 of the flutes, referred to as the web 34, forms the core of the drill bit body. The web 34 substantially contributes to the torsional and flexural strength of the bit but conventionally it is substantially smaller in cross section than the extending land 16 thus limiting the stress and bending resistance of the drill bit 10.

The present invention provides a more robust surgical drill bit that minimizes the buildup of heat in the bone and reduces the chance that necrosis of the bone will occur. In addition, surgical drill bits are usually of small diameter and the pressure that must be applied during drilling can result in bending or breaking the drill bit. This results in an interruption of the operation while the bit is replaced and in some cases the broken portion of the drill bit in the bone cannot be removed. The drill bit of the invention is more resistant to flexural and torsional stress and can more readily withstand stresses encountered during drilling and therefor is less likely to bend or break during a procedure.

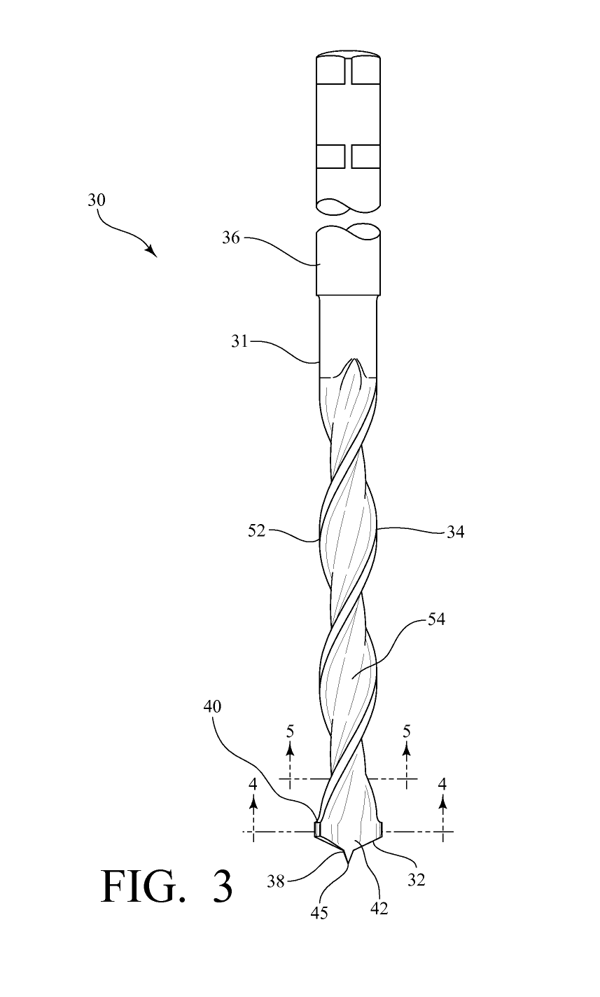

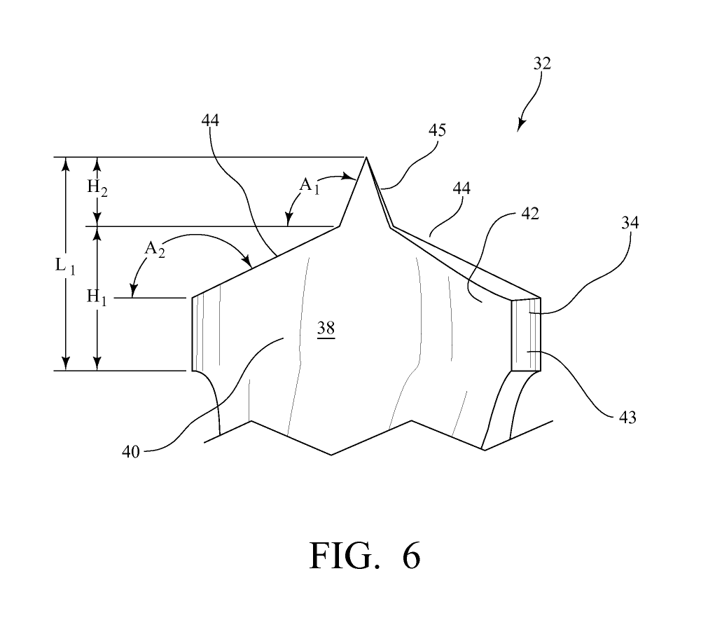

Referring to FIG. 3 and FIG. 6 there is shown a surgical drill bit 30 in accordance with the invention. The drill bit comprises an elongated cylindrical body 31 comprising three sections; a drill section 32, a midsection 34 and a drive transmission shank 36 adapted for attachment to a power tool of conventional design, not shown.

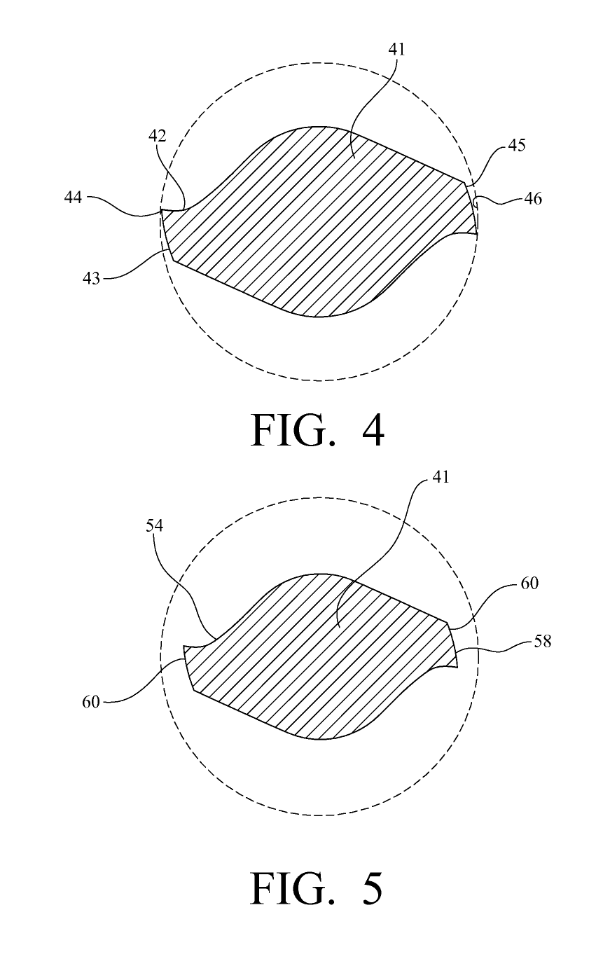

As illustrated, the drill section 32 includes an opposed pair of flutes 40 that helically extend along the length of the drill section and a drill point 38 at its distal end. Each flute 40 defines a land 42 that extend helically, outwardly from an enlarged core 41. As most clearly shown in FIG. 4 the extending outer surface 43 of the land 42 forms a cutting edge 44 and a trailing portion 45. The trailing portion 45 is biased inwardly with respect to the cutting edge 44 to provide body clearance 46 between it and the bore wall. The cutting edge 44 of the land 42 defines the outer diameter of the drill bit 30 and the resulting diameter of the bore formed by the drill bit.

Referring to FIG. 6 the distal end of the drill section 32 defines a drill point 38. The drill point 38 comprises cutting edges 44 sloping at an angle from the distal tip of the drill section 32 to the edge 43 of a respective land 42. The angle A₂ defined between the edges 44 forming the drill point 38 and the edge 43 of the land is a matter of choice and may range from about 90° to about 180°. The wider the point angle the shorter the cutting edge at the drill point 38 and therefore less contact with the bone and less heat generation. A point angle of between 100° and 170° is preferred for drilling bone. The drill point 38 may comprise any conventional drill point configuration and the prior art is replete with various configurations depending on the material being drilled. For the purposes of the invention a split point configuration is preferred as generating the least amount of heat.

A major source of heat buildup is due to friction between the outer surface 43 of the land and the material being drilled. Conventionally, with surgical drills, the outer surface of the lands bear against the inner face of the bore along the entire length of the bit producing heat due to friction. In addition, debris from the drilling procedure can accumulate in the body clearance between the trailing surface of the land and the wall of the bore creating more friction and heat buildup. Since the function of the drill section 32 and the drill point 38 is to provide the cutting action of the bit 30, the axial length L₁ can be kept to a minimum, preferably between 1 to 2 times the outside diameter of the drill section. Direct contact between the outer surface 43 of the flutes 40 and the wall of the bore is kept to a minimum.

Although the drill section 32 is illustrated with a pair of flutes 40 it will be understood the drill section may comprise a single flute or more than 2 flutes for example, 3 or 4 flutes.

As shown in FIG. 3 flutes 52 that extend along the length of the midsection 34 have a smaller outside diameter than the drill section 32. As most clearly shown in FIG. 5 flutes 52 comprise lands 54 that extend from the core 41 to a lesser extent than lands 42 of the drill section 32 so that the outer surfaces 58 define a larger body clearance 60 for removal of bone chips without unduly producing friction by bone chips becoming compacted between the surface 58 and the wall of the bore. Good results are achieved when the outside diameter of the midsection 34 is reduced about 5%-10% as compared to the outside diameter of the drill section 32. The larger body clearance 60 does not interfere with the chip removing function of the midsection 34. The flutes 52 may be integrally formed with the flutes 40 or may be separately formed. Additionally, the number of flutes 52 on the midsection 34 may be different than the number of the flutes 40 on the drill section 32. For example it may be advantageous to provide the midsection 34 with four flutes 40 to aid in removal of bone cuttings while providing the drill section 32 with 2 or 3 flutes in order to minimize friction during the drilling procedure.

In accordance with the invention the drill bit 30 has improved flexural and torsional strength. Referring to FIG. 4 and FIG. 5, in cross section the drill bit body 31 comprises a web 41 that is substantially enlarged while the width of the lands 42 and 54 at their outer surfaces, 43 and 58 respectively, is reduced. In accordance with the invention, the web 41 comprises between about 40% and 70% of the diameter of the drill bit body 31 and decreases gradually to form the lands 42 and 54 respectively. The land width at the outer surface 44 and 58 is reduced since land width is not essential to the strength of the drill bit 30. In particular the reduced width of the lands 42 and 54 reduces the outer surface area of the lands 42 and 54. In addition the reduced diameter of the mid-section 34 provides additional body clearance 60 for chip removal while permitting the reduced land width. In this manner there is less area in contact with the bore wall and bone chips resulting in a reduction of friction and resultant heat buildup. With conventional surgical drill bits, such as shown in FIG. 2, where the flutes are of equal diameter along the entire drill bit, the width of the lands must be wide enough to provide flexural strength to the drill bit. In addition the width of the lands 16 provide land edges to form a sufficient trailing surface area 19 for receiving and removing bone chips

The enlarged web 41 substantially increases torsional and flexural strength of the drill bit 30. Calculations show that for equal cross section and equal material drill bit 30 with the increased web 50 has up to 20% lower normal stress (bending) and over 30% lower shear stress (torsion).

Centering and maintaining the position of the drill bit 30 on bone can be difficult especially with the wider preferred point angle. As illustrated in FIG. 6 a concentric tip 45 is formed on the drill point 38. The axial height H₂ of the concentric tip 45 is preferably about ⅓ of the axial height H₁ of the drill point 38. The concentric tip 45 is conical in shape and defines an angle A1 of between 50° and 80°. The concentric tip 45 serves to aid in maintaining the position of the drill bit during the start of the drilling procedure to reduce “bit walking” on the bone being drilled.

Several factors have to be considered when selecting the material for the surgical drill bit in view of the medical applications for which the drill bit will be used. Martensitic stainless steel (410 or 420) is preferred for the surgical drill bit. Martensitic stainless steel is preferred because it is resistant to corrosion, can be easily machine in annealed condition possesses a high ultimate tensile strength and very good edge-keeping ability in the hardened condition.

It is preferred that the entire drill bit 30 be formed from hardened martensitic steel to reduce manufacturing costs without sacrificing the effectiveness of the bit. However, the drill section 32 can be formed of a different material such as carbide. Likewise, the midsection 34 and drive transmission shank 36 may be formed of a different material such as a high strength plastic while the drill section is hardened martensitic steel.

Surface condition of the surgical drill bit is a consideration in reducing friction and heat buildup in bone. A smooth, low coefficient of friction, surface results in lower friction and, as consequence, a lower temperature increase. The best surface condition can be achieved by a polishing process but processing times and associated costs are unjustifiably high. Fine grinding of the external diameter and flute offers a better cost/benefit combination. Some products are electro polished but this process, while assuring a low surface roughness, rounds off the cutting edges thus reducing the cutting ability. Coating the drill bit with a material having a low coefficient of friction has been commonly used with conventional drill bits. Thus, it is preferred to coat the surgical drill bit of the invention with a suitable coating to reduce the coefficient of friction.

Some of the existing drill bits are offered with a titanium nitride (TiN) coating. TiN is an extremely hard ceramic material and is applied as a layer of less than 5 micrometers thickness. In a preferred embodiment of the invention a-C:H coating, which is a diamond-like carbon layer with very high density, is applied over at least the drill section 32. Compared to TiN, a-CH is slightly harder (2500 vs. 2300 HV), is thinner (2-3 vs. 5 micrometers) and, most important, has a friction coefficient on steel of 0.1-0.2 vs. 0.4 for TiN or 0.5-0.8 of steel. The lower friction force between this coating and the material being drilled results in lower heat generation and, thus, lower increase in temperature of the material.

From the foregoing description and drawings it will be seen that the present invention provides an improved drill bit for drilling bone that results in lower temperature buildup in the bone for a given period of time during the drilling procedure. While the embodiments of the invention have been disclosed herein have been particularly shown and described with reference to the exemplary embodiments thereof, those of ordinary skill in the art will understand that various changes may be made in the form and details herein without departing from the spirit and scope of the disclosure and the appended claims. Those of ordinary skill in the art will recognize or be able to ascertain many equivalents to the exemplary embodiments described specifically herein by using no more than routine experimentation. Such equivalents are intended to be encompassed by the scope of the present disclosure and the appended claims.

What is claimed is:

1. A surgical drill bit consisting of an elongated cylindrical body having a core, an outer surface and a distal and proximal end portion, said distal end portion defining a drill section, said proximal end portion defining a transmission section for attachment to a power device and a midsection disposed there between, said drill section and said midsection each having at least one flute comprising a land radially, helically extending from said core, the extending edge of said land of said drill section defining a cutting edge and forming the outside diameter of said surgical bit, the extending edge of said land of said midsection defining an outside diameter less than that of said drill section.

2. The surgical bit of claim 1 wherein said drill section comprises an opposed pair of helical flutes extending axially the length thereof.

3. The surgical bit of claim 1 wherein said midsection comprises an opposed pair of helical flutes extending axially the length thereof.

4. The surgical drill bit of claim 1 wherein said flute of said drill section and said midsection comprises in cross section opposed lands extending radially from an enlarged central web and having an outer surface defining the cutting edge of said lands.

5. The surgical drill bit of claim 4 wherein said enlarged central web is at least forty percent of the outside diameter of said drill section thereby to provide improved flexural and torsional strength to said surgical drill bit.

6. The surgical drill bit of claim 4 wherein the cross section of said enlarged web is between about 40 percent to about 70 percent of the outside diameter of said drill section.

7. The surgical drill bit of claim 1 wherein the outer surface of said land on said drill section comprises a cutting edge.

8. The surgical drill bit of claim 1 wherein the distal end of said drill section comprises a drill point, said drill point comprising opposed cutting edges extending from the tip of said drill point to the outer edge of a respective land.

9. The surgical drill bit of claim 6 wherein said opposed cutting edges form an angle of between about 90° and about 180°.

10. The surgical drill bit of claim 6 wherein said opposed cutting edges form an angle of between about 100° and about 170°.

11. The surgical drill bit of claim 6 wherein a concentric tip is formed on the distal end of said drill point.

12. The surgical drill bit of claim 7 wherein said concentric tip is conical and defines an angle of between about 50° and 80°.

13. The surgical drill bit of claim 6 wherein the axial height of said conical tip is about one third of the axial height of said drill point.

14. The surgical drill bit of claim 1 wherein the axial length of said drill section is between about 1 to about 2 times the outside diameter of said drill bit at said drill section.

15. The surgical drill bit of claim 1 wherein the outside diameter of said midsection is at least about 5 percent less than the outside diameter of said drill section.

16. The surgical drill bit of claim 14 wherein the outside diameter of said midsection is between about 5 percent and 10 percent less than the outside diameter of said drill section.

17. The surgical bit of claim 1 wherein said flute of said drill section and said midsection are integral and extend from said drill section to the end of said midsection at said transmission section.

18. The surgical drill bit of claim 1 wherein said flute of said drill section is separately formed from the flute of said midsection.

19. The surgical drill bit of claim 1 wherein said drill section and said midsection comprise 3 helical flutes extending axially the length thereof.

20. The surgical drill bit of claim 1 wherein the number of flutes on the drill section is different than the number on the midsection.

21. The surgical drill bit of claim 1 wherein the surface is a low coefficient of friction surface provided by a coating of a diamond like carbon layer.
